(19) United States
(12) Patent Application Publication
(10) Pub. No.: US 2020/0175197 A1
(43) Pub. Date: Jun. 4, 2020

(54) SYSTEM AND METHOD FOR ENABLING A MULTI-LOCATION DATA STORAGE AND RETRIEVAL SYSTEM

(71) Applicant: BRIGHTSQUID DENTAL LTD., Calgary (CA)

(72) Inventors: JASON MYERS, Calgary (CA); FRANCIS CHEW, Calgary (CA); ROHIT JOSHI, Calgary (CA); DEREK SCHERGER, Calgary (CA)

(21) Appl. No.: 16/780,716

(22) Filed: Feb. 3, 2020

Related U.S. Application Data
(63) Continuation of application No. 15/552,596, filed on Aug. 22, 2017, now abandoned.
(60) Provisional application No. 62/120,113, filed on Feb. 24, 2015.

(51) Int. Cl. G06F 21/62; G06F 16/958; G06F 16/95; G06F 21/33; G06F 12/14; G06F 3/06; G16H 10/60

(52) U.S. Cl. CPC G06F 21/6245; G06F 16/972; G06F 16/95; G06F 21/335; G06F 12/1408; G06F 21/78; G06F 3/0637; G06F 3/0622; G16H 10/60; G06F 2212/1052; G06F 3/067

(57) ABSTRACT

A system and method for storing data in multiple locations on the basis of rules maintained by the system. The invention can enable data management, collaboration of data usage between users and the storage of data. The invention can be used for just-in-time location, retrieval, aggregation and delivery of a view of information that may not result in the information being moved from or stored other than from the approved location. Optionally, further assurance of data location may be periodically provided by a location audit service.

### FIELD OF THE INVENTION

The present invention relates to a system and method for storing data in multiple locations on the basis of rules maintained by the system. When requested by a user, the invention would be used for just-in-time location, retrieval, aggregation and delivery of a view of the information that does not result in the information being moved from or stored other than from the approved location. Optionally, further assurance of data location may be periodically provided by a location audit service.

### BACKGROUND OF THE INVENTION

Information stored as data within a computer system may be stored in the immediate vicinity of the computer and the information user. There are economic, security and logistic advantages to storing computer data remote from the computer. There are times when stored data is created by one user but it is to be accessible by multiple users, potentially in different locations.

Internet based data storage systems are typically convenient approaches to data storage, and can be readily configured for collaborative use by multiple users in different locations. The physical location of the stored data is not generally apparent for such Internet based data storage. This makes them unsuitable for applications where data location is subject to legal, regulatory, confidentiality, privacy, security or other business requirements. Examples of such data include medical records, financial data, legal records, governmental data, military information, trade secrets and security data.

There are scenarios where a user may benefit from comparing specific aspects of other data without wishing to assume the liabilities associated with having a copy of the data. For example, a doctor may wish to compare the treatment plan for one of their own patients with the plan and outcome of a colleague's patient. The doctor does not wish to become owner or make a copy of the colleague's patient's medical record.

### SUMMARY OF THE INVENTION

The present invention is generally directed to a system and method for storing data in multiple locations on the basis of rules maintained by the system. The invention can enable data management, collaboration of data usage between users and the storage of data. At the request of a user, the invention can be used for just-in-time location, retrieval, aggregation and delivery of a view of information that may not result in the information being moved from or stored other than from the approved location.

In accordance with an aspect of the present invention a method is provided for enabling data management, collaboration of data usage between users and data storage including requiring user authentication for allowing access to a user, permitting the user to manage data and to share data with one or more additional users, permitting access of the data to one or more of the additional users, determining a location for storage of the data, and storing the data at the storage location.

In accordance with another aspect of the present invention, a method is provided for enabling dynamic patient record collaboration and storage of patient record data to a requisite jurisdiction, the method comprising requesting and receiving authentication information from a first user, receiving data from the first user with respect to a patient, the data including the patient address, creating a data record based on the data and storing the data record in a default or user-selected storage location, generating a random ID corresponding to the data record, receiving a request by the first user to share the data record with a second user, requesting and receiving authentication information from the second user, retrieving the data record from the storage location and displaying the data record to the second user, and deleting all temporary data.

In accordance with another aspect of the present invention, a system is provided for enabling data management, collaboration of data usage between users and data storage comprising: a login portal for receiving an access request from a first user for allowing access to the system, the login portal adapted for requiring user authentication and providing an interface for a first user to manage data or share data with one or more additional users; a data location means for locating and retrieving data and determining a location for data storage; and storage means, for providing storage for the data and user authentication information, the storage means in communication with the data location means.

In accordance with another aspect of the present invention, a system is provided for enabling data management, collaboration of data usage between users and data storage comprising: a login portal for requesting and receiving authentication information from a first user, the login portal adapted for providing an interface for a first user for receiving data, or receiving a request by the first user to share data with one or more additional users; a location rules server for locating, retrieving data and assembling data to present it in a consolidated temporary view, and determining a location for data storage, the location for data storage determined by default with an option to select optional storage locations; and one or more data storage servers, for providing storage for the data and user authentication information, the data storage servers in communication with the location rules server.

### BRIEF DESCRIPTION OF THE DRAWINGS

Referring to the drawings, several aspects of the present invention are illustrated by way of example, and not by way of limitation, in detail in the figures, wherein:

FIG. 1 shows an example of the system architecture with multiple users and multiple Data Storage Servers.

FIG. 2 shows an example process flow chart for the creation of a data record by a User A and the viewing and editing by a User B.

FIG. 3 shows an example of a screenshot of a collaboration between two users which includes data from two physical locations.

### DESCRIPTION OF THE VARIOUS EMBODIMENTS

The detailed description set forth below in connection with the appended drawings is intended as a description of various embodiments of the present invention and is not intended to represent the only embodiments contemplated by the inventor. The detailed description includes specific details for the purpose of providing a comprehensive understanding of the present invention. However, it will be apparent to those skilled in the art that the present invention may be practiced without these specific details. To the extent that the following description is of a specific embodiment

For various reasons, sensitive information may be required to be stored in geographic locations where it originates. Cloud based server hosting systems will not typically discriminate the location of data storage, but rather, determine the location of storage based on the convenience and economies inherent to the cloud provider. Generally, viewing information from these cloud bases systems includes multiple caches, storage and duplication of data which is likely poorly controlled or not controlled at all by the end user.

The present invention aims to address the various concerns of data creators when storing their data on cloud networks where the storage location of data is uncertain. In the event that the cloud provider allows the location of the data to be specified, it may be more appropriate to have the cloud system store data to different physical locations automatically or as specified by the user who creates the data. The reasons that the data creators may want to select the location of their data storage may be associated with legal, regulatory, confidentiality, privacy, security or other business requirements. In addition to the data storage, the meta-data associated with the data as well as the contents of any messaging are preferably stored in the same jurisdiction as the originating data. A method that is used to identify and control the location that the data is created is described herein, and a subsequent method that is used to retrieve, view and modify the data stored in that location is also described herein.

In other circumstances, businesses may determine that they require their data be stored in specific locations. There are various considerations for business data storage, including the physical location of the data. The present invention may help enable the originator of the business information to control and verify the location of their data storage.

The present invention is configured such that data may be assembled from multiple sources, creating a temporary view of the data in a way which does not rely on information being moved, duplicated or stored other than in the approved location. In the event the data is modified, the modifications are stored at the same location as the originating data.

In a typical scenario, a group of users intend to share information deemed to be private or confidential. The originator of the information seeks to ensure that the information is stored in a location of his choosing. By default, the data is stored in the location where the information has originated. In the event a Data Storage Server is not located in the same location as the originator, the data is stored in a default location as determined by business requirements. Once the data has been created and stored on the server, the data originator has the option to invite other users to add or modify to the data set. All changes to the data are stored in the location of the original data. In the event the originator needs to change the location of data storage, they may have the option to do so.

Within the bounds of heath care data storage, for example, there are regulatory and policy reasons for patient data to be stored in the location of a (a) patient's residence or (b) where the health care service is provided. Health information exchange for the purposes of collaboration, consultation, and/or education is subject to legal restrictions on where the health care data can be stored. Several laws in the USA, Canada and EU restrict the location where it is acceptable to store patient information, hampering the ability to share patient treatment details when necessary for treatment delivery or education. Many systems exist for managing data within one location, for example patient record management software. These systems are fundamentally unable to allow sharing of such data between collaborators in different jurisdictions. To solve this problem, it is important to store the patient data in their current legal jurisdiction while still enabling the sharing of the contents of the patient file across jurisdictions.

For the purposes of data storage, a system according to an embodiment of the present invention automatically determines the location to store the data based on a first identifier associated with the originating data. The first identifier may be, for example, the patient address. When collaboration between users in multiple locations is required, the system assembles the patient record and enables the collaboration with a temporary view of the data. When the collaboration is complete, the system stores any further modifications back to the location of the original patient data storage. Where collaborations include data from multiple locations, the edits themselves are stored in the same location as the data to which they are associated. The present invention enables dynamic patient record collaboration, independent of jurisdiction, and also enables the storage of the information back to the requisite jurisdiction.

In one embodiment, the system may comprise components that perform the following basic functions. The three functions are: (i) the Login Portal, (ii) the Location Rules Server, and (iii) one or more Data Storage Servers. Although the Login Portal and the Location Rules Server may be served from any location, the Data Storage Servers are located in one or more of the data centers which may have specific known and controlled locations. These functions may be executed by software, which may or may not be on separate servers.

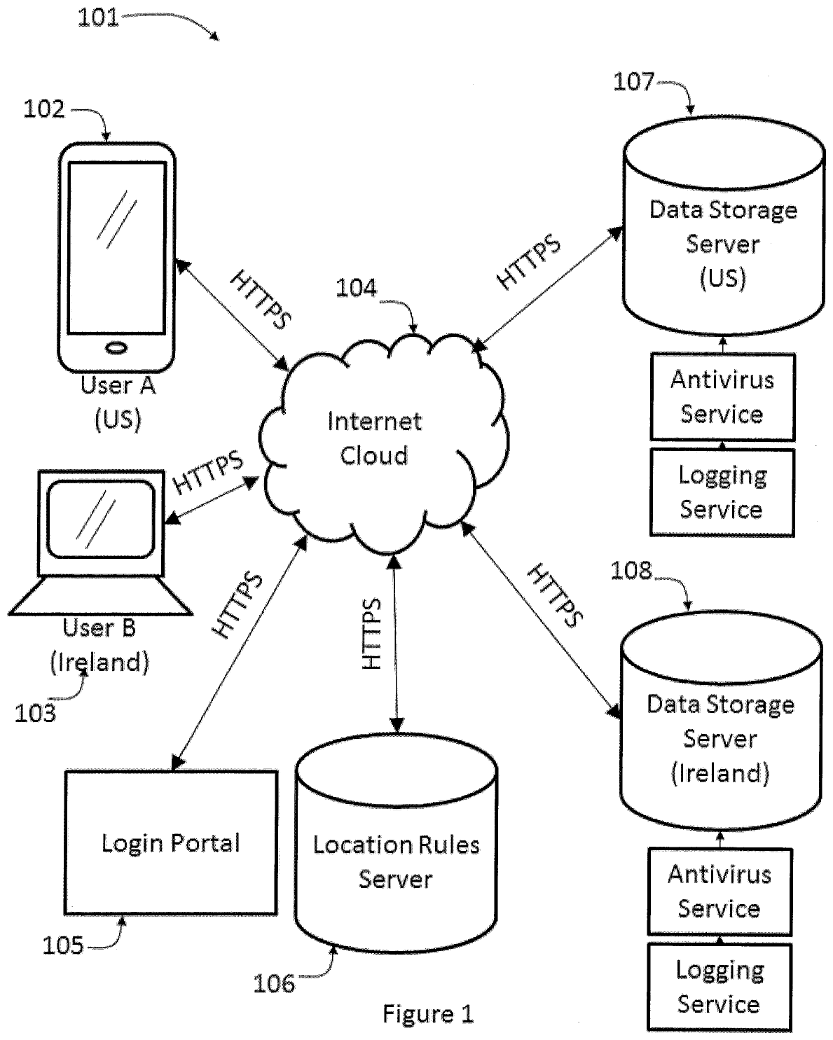

FIG. 1 illustrates one example of the architecture of the system (101), although number of servers and number of locations are expected to vary from a single instance to multiple instances according to requirements of the system. Each of the users will join through their own device, which could be a personal computer, tablet or smart phone. For example, User A located in the USA and connecting to the system with a smart phone, (102) and User B located in Ireland and connecting through a personal computer, (103). Each of the devices will be equipped with a processor, storage system and will have an input system to enter commands. Each of the devices will be equipped to communicate with the Internet, (104) through HTTP protocol (Hyper Text Transfer Protocol) or other similar protocols to gain access to the system.

The Login Portal, (105) is preferably a central cloud application server. The Login Portal receives a request from the user's device which forces login to the system as well as provides an interface to upload information or share the information with other collaborators. The Login Portal communicates with the Data Storage Servers, (107) and (108) through HTTPS requests and orchestrates all registration, user access and permissions from the Data Storage Servers. In a medical example, this orchestration of information would include all patient information, messages, attachments and treatments and other data elements which have been identified as confidential patient information.

The Location Rules Server, (106) is aware of the multiple Data Storage Servers and regions. Based on the business rules, the Location Rules Server determines where the data is to be stored. It is likely that no confidential data is available at the location of the Location Rules Server or at the location of the Login Portal, but may be permissible based on requirements. When locating data, the Location Rules Server may interrogate all of the Data Storage Servers immediately and asynchronously, or based on requirements may interrogate the Data Storage Servers in a serial manner and on a timed basis. Alternatively, an index of the data may contain necessary location information and may be anonymized such that it may be arbitrarily located. During the data retrieval processes, the Location Rules Server will send out requests to each of the Data Storage Servers, to locate, retrieve and correctly assemble the information from the Data Storage Servers to present it to the user in a consolidated temporary view. For example, if a data collaboration session pulls data from multiple Data Storage Servers, the information will be assembled as it arrives from the Data Storage Servers for presentation to the end user.

It is possible for the creation of multiple Login Portals, each serving a different market segment. In the event multiple Login Portals are created, user management and permissions would be applied by the Login Portal. Each Login Portal would communicate with the Location Rules Server through an API. Optionally, separate Login Portals could operate independently providing separate groups of users distinct multi-location data storage systems. These separate Login Portals could make use of the same system architecture and server hardware and all associated with the same Location Rules Server. For example a system which is configured to provide collaborative medical record management in multiple locations, could also be configured to provide collaborative financial data storage in multiple locations.

Each of the Data Storage Servers stores information on the basis of the rules found in the Location Rules Server. Optionally, data uploaded to the Data Storage Server will be processed through an antivirus program to prevent distribution of viruses and malware. For quicker access to the information, the Data Storage Server may also index all patient data such that it can be searched by an authorized user. Finally, it may be advantageous for the Data Storage Server to log all user activity to provide an audit trail of any interaction with data, although this would depend on the specific requirements. In a medical example, including confidential patient information as well as confidential user information. All data, including all metadata regarding the patient information and user authentication is stored in the Data Storage Server.

Each data center may be a cloud storage server and may be located in a different geographical location. Preferably, all data stored on cloud storage servers are encrypted, although the specifics would depend on usage requirements.

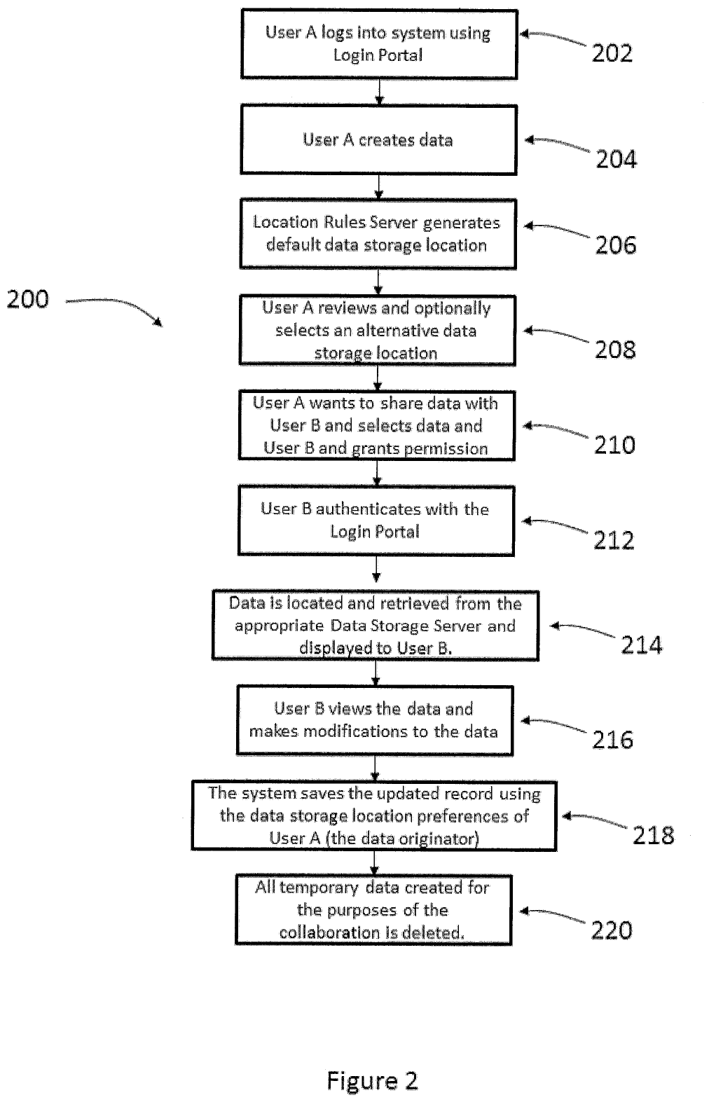

For the purposes of clarity, FIG. 2 illustrates the method by which an authorized user is able to collaborate with another authorized user. There could be multiple collaborators in this example with data stored in multiple locations. With reference to FIG. 2, there is provided a method (200) for enabling dynamic patient record collaboration, independent of location, and enabling the storage of the information back to the requisite location. The system includes a Login Portal, (202) by which a user can access the system. The Login Portal includes user authentication for example using password or two factor authentication.

When a user, for example User A, creates a data record, (204), the system provides a default storage location for the data, (206), for example, based on the address of the patient. The system provides User A with the option to select alternative storage locations, if for example a patient is located in a different country, (208).

The system creates a single version of the data record on the Data Storage Server in the selected jurisdiction. User A can continue to access this record and edit it as necessary. Each time User A is authenticated by the Login Server. If required, each edit is recorded by the logging service of the Data Storage Server for audit purposes. Other users cannot, by default, access the data record. Every time User A wants to access the data, the data is assembled from the Data Storage Server.

In the event that User A is collaborating with User B, (210), for example about patient X, the system provides an interface whereby User A can select the data record of patient X, to be shared and the user to whom permission will be granted, User B. Optionally, such permission can be time limited, and is not transferable. The information shared by User A can be read/write restricted and the system gives no ability for User B to copy or delete the data.

Subsequently, when User B accesses the system through the Login Portal, (212), the system includes User A's shared data of patient X in the collection of records available to User B to view and modify. In the case where a collaboration includes an ongoing message thread, the system will display to User B the thread by assembling the data from different locations in an organized structure, for example chronologically.

The system locates and retrieves the data from the geographical location and displays the data to User B (214). The system allows User B to view and modify the data (216). The system also allows User B to save the modified data to the same geographical location as previously selected by User A for the original data (218). The system then deletes all temporary data created for the purpose of the collaboration between Users A and B (220).

For the purposes of auditing all transactions on the system, a separate logging service may be included associated with each Data Storage Server for tracking all interactions with respect to the data.

For medical records, typically the patient is the only person to instigate the relocation of their own medical data, if for example they have relocated to a different country. In this case, the patient requests the relocation of their data from the person or institute that originally created the data, which may be, for example, their original doctor in their original country. The patient also specifies their new doctor in their new country as the recipient of the data record. The original data creator, i.e. the original doctor, grants data duplication and/or relocation permission to the recipient, i.e. the new doctor. In most cases, patient data will be duplicated for the new doctor in the new location with the existing data remaining in the original location archived for regulatory reasons. Only upon specific request by the patient will the original data be deleted and entirely relocated to the new location. This “push-relocation” method gives assurance to the original data creator that the data cannot be duplicated or relocated without their consent. For medical records, this control is often necessary for regulatory compliance.

Data location integrity may be periodically checked by a separate auditing system. This may, for example, be an automatic system which periodically checks that records are accessible to the Login Portal, but are physically stored in the location specified by the Location Rules Server. This may include tracing the routing of the data and/or using IP address based methods.

Optionally, the functions of the Login Portal and Location Rules Server may be in a single system, multiple systems in a single location or distributed over a number of connected servers. This may be useful to improve system performance where a majority of collaborations are largely within the same geographical location and the associated data is also likely to be stored in the same geographical location.

In a further optional embodiment, users of the system may limit the permissions that they grant to other users. Such limits may include, for example, time limits, access to specific data elements but not to an entire record, geographical access limits, permission or restrictions for further reuse, commercial or non-commercial use, etc.

For some medical collaborations such as research trials, access to medical data may be further controlled by ethics or confidentiality agreements. This system may include such agreements in the Login Portal to help ensure compliance with legal restrictions for data sharing. If a data sharing request as part of a research trial is made, but where the data recipient is not a signatory to the agreement, then the system may either decline data sharing or provide a copy of the required ethics or confidentiality agreements for the recipient's execution before sharing the data.

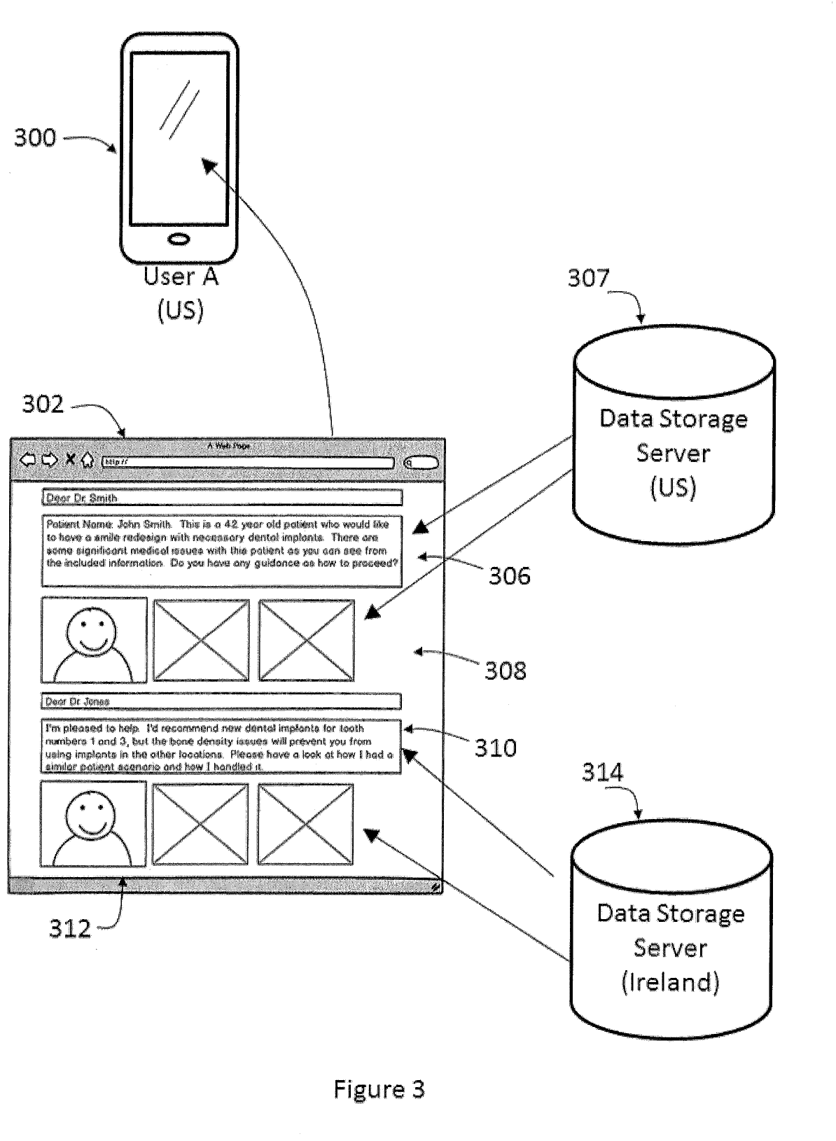

FIG. 3 has been included for the purposes of illustrating a sample graphical user interface is illustrated wherein a User A is corresponding with a User B. In this example, User A is located in the USA (300) and User B is located in Ireland (not shown). For legal reasons, User B's patient data has to stay in the European Union, including any annotations and treatment recommendations. Similarly, since User A is located in the USA, the data relating to the sample patient that he uses to describe a similar treatment has to be stored in the USA.

On User A's device, (300), the graphical user interface displays the interaction, (302), in this case organizing the messages chronologically. The message includes patient data retrieved from the Data Storage Server in the USA, (307). This data includes text, (306) and images (308). In addition, this message includes patient data retrieved from a Data Storage Server in Ireland, (314). This data includes text (310), and images (312). Upon the completion of this interaction, all of the information will be stored in the location of the original patient information.

The previous description of the disclosed embodiments is provided to enable any person skilled in the art to make or use the present invention. Various modifications to those embodiments will be readily apparent to those skilled in the art, and the generic principles defined herein may be applied to other embodiments without departing from the spirit or scope of the invention. Thus, the present invention is not intended to be limited to the embodiments shown herein, but is to be accorded the full scope consistent with the claims, wherein reference to an element in the singular, such as by use of the article “a” or “an” is not intended to mean “one and only one” unless specifically so stated, but rather “one or more”. All structural and functional equivalents to the elements of the various embodiments described throughout the disclosure that are known or later come to be known to those of ordinary skill in the art are intended to be encompassed by the elements of the claims. Moreover, nothing disclosed herein is intended to be dedicated to the public regardless of whether such disclosure is explicitly recited in the claims.

The above-described embodiments of the invention are intended to be examples only. Alternations, modifications and variations can be effected to the particular embodiments by those of skill in the art without departing from the scope of the invention.

What is claimed is:

1. A method for enabling data management, collaboration of data usage between users and data storage comprising:
requiring user authentication for allowing access to a first user;
permitting the first user to manage data and to share data with one or more additional users;
permitting access of the data to the one or more additional users;
determining a location for storage of the data; and
storing the data at one or more storage locations.

2. The method of claim 1, further comprising locating and retrieving existing data from one or more storage locations and displaying the data to the first user after allowing access to the first user.

3. The method of claim 2, further comprising aggregating the existing data retrieved from the one or more storage locations so as to provide a consolidated display of the data.

4. The method of claim 1, wherein managing data comprises creating original data, viewing existing data, adding to existing data, or modifying existing data.

5. The method of claim 4, wherein modifications and additions to existing data are stored in the same storage location as original data.

6. The method of claim 4, wherein the storage location is determined by default or by selection by the first user.

7. The method of claim 6, wherein the default storage location is the original data storage location or is determined by business requirements.

8. The method of claim 1, wherein the one or more additional users are permitted data access by request of the first user.

9. The method of claim 8, further comprising requesting and receiving authentication information from the one or more additional users prior to permitting access to the one or more additional users.

10. The method of claim 9, wherein access of the data to the one or more additional users comprises viewing of existing data, modifying of existing data, or adding to existing data.

11. The method of claim 1, further comprising placing restrictions or limitations to the access of the data to the one or more additional users.

12. The method of claim 11, wherein the restrictions or limitations placed are based on time, specific data elements, geographic access, commercial or non-commercial usage, ethics requirements or confidentiality requirements.

13. The method of claim 10, wherein the one or more additional users are prohibited from duplicating, transferring or deleting the data.

14. The method of claim 1, further comprising processing the data stored through an antivirus program.

15. The method of claim 1, further comprising auditing user activity for providing an audit trail of any user interaction with the data.

16. The method of claim 1, further comprising encrypting the data prior to storage.

17. The method of claim 1, further comprising deleting any temporary data.

18. The method of claim 1, wherein the data comprises health care data.

19. The method of claim 18, wherein location for storage of the data is determined by patient residence or location of health care service provider.

20. The method of claim 4, wherein location for data storage is based on a first identifier associated with the original data.

21. The method of claim 20, wherein the first identifier is patient address.

22. A method for enabling dynamic patient record collaboration and storage of patient record data to a requisite jurisdiction, the method comprising:
requesting and receiving authentication information from a first user;
receiving data from the first user with respect to a patient, the data including the patient address;
creating a data record based on the data and storing the data record in a default or user-selected storage location;
generating a random ID corresponding to the data record;
receiving a request by the first user to share the data record with a second user;
requesting and receiving authentication information from the second user;
retrieving the data record from the storage location and displaying the data record to the second user; and
deleting all temporary data.

23. The method of claim 22, wherein the data includes an address of the patient, and wherein the default storage location is based on the address.

24. The method of claim 23, further comprising allowing the second user to modify the data record and saving the modified data record at the storage location.

25. A system for enabling data management, collaboration of data usage between users and data storage comprising:
a login portal for receiving an access request from a first user for allowing access to the system, the login portal adapted for requiring user authentication and providing an interface for a first user to manage data or share data with one or more additional users;
a data location means for locating and retrieving data and determining a location for data storage; and
storage means, for providing storage for the data and user authentication information, the storage means in communication with the data location means.

26. The system of claim 25, wherein the storage means comprises one or more data storage servers.

27. The system of claim 26, wherein the data storage servers are located in different geographical locations.

28. The system of claim 26, wherein the data stored on the one or more data storage servers is encrypted.

29. The system of claim 26, wherein the data location means comprises one or more location rules server.

30. The system of claim 29, wherein each data storage server stores data on the basis of rules found in the location rules server.

31. The system of claim 25, wherein management of the data by a first user comprises creating original data, uploading and viewing existing data, modifying existing data, or adding to existing data.

32. The system of claim 26, wherein the storage location for a user to store data is determined by default with the first user provided with an option to select optional storage locations.

33. The system of claim 32, further comprising auditing means for providing an audit trail for interactions with the data by a user.

34. The system of claim 33, wherein the auditing means is provided by the data storage server.

35. The system of claim 30, wherein the login portal permits the first user to select the data to be shared with the one or more additional users.

36. The system of claim 35, wherein permission to share data with the one or more additional users is restricted or limited on the basis of time, specific data elements, geographical access, commercial or non-commercial use, ethics requirements, confidentiality requirements, transferability, duplication of the data, or deletion of the data.

37. The system of claim 35, wherein the data shared with the one or more additional users is read/write restricted.

38. The system of claim 29, wherein the location rules server assembles the data retrieved for providing a consolidated view for a user.

39. A system for enabling data management, collaboration of data usage between users and data storage comprising:
a login portal for requesting and receiving authentication information from a first user, the login portal adapted for providing an interface for a first user for receiving data, or receiving a request by the first user to share data with one or more additional users;
a location rules server for locating, retrieving and assembling data for presentation in a consolidated temporary view, and determining a location for data storage, the location for data storage determined by default with an option to select optional storage locations; and
one or more data storage servers, for providing storage for the data and user authentication information, the data storage servers in communication with the location rules server.

40. The system of claim 39, wherein the data includes an address of the patient, and wherein the default storage location is based on the address.

41. The system of claim 39, wherein the location rules server retrieves data from a storage location for display to the one or more additional users and allowing the one or more additional users to modify the data and save the modified data at the storage location.
